(19) United States
(12) Patent Application Publication
(10) Pub. No.: US 2024/0117434 A1
(43) Pub. Date: Apr. 11, 2024

(54) METHODS AND RELATED ASPECTS FOR DETECTING DISEASES, CONDITIONS, OR DISORDERS IN SUBJECTS USING EXTRACELLUAR VESICLES

(71) Applicant: THE JOHNS HOPKINS UNIVERSITY, Baltimore, MD (US)

(72) Inventor: Samarjit DAS, Eldersburg, MD (US)

(73) Assignee: THE JOHNS HOPKINS UNIVERSITY, Baltimore, MD (US)

(21) Appl. No.: 18/277,186

(22) PCT Filed: Feb. 18, 2022
(86) PCT No.: PCT/US2022/017083

§ 371 Date: Aug. 14, 2023

Related U.S. Application Data
(60) Provisional application No. 63/150,907, filed on Feb. 18, 2021.

(51) Int. Cl. C12Q 1/6883; B01L 3/00; C12Q 1/6804; C12Q 1/70

(52) U.S. Cl. CPC C12Q 1/6883; B01L 3/502761; C12Q 1/6804; C12Q 1/701; B01L 2200/028; B01L 2200/0647; B01L 2300/0636; B01L 2400/0436; C12Q 2600/178

(57) ABSTRACT

Provided herein are methods of detecting a disease, condition, or disorder in a subject. In some embodiments, the methods include obtaining a set of ribonucleic acid (RNA) molecules from a population of exosomes in a biological sample obtained from the subject and detecting a plurality of target RNA molecules corresponding to a selected set of cell-specific exosomal RNA molecules in the set of RNA molecules to generate a target RNA molecular profile for the sample. In some of these embodiments, the methods also include determining that the target RNA molecular profile substantially matches a reference RNA molecular profile that correlates with the disease, condition, or disorder in a subject and/or using at least one algorithm that predicts a likelihood that the target RNA molecular profile correlates with the disease, condition, or disorder in a subject. Related biosensor devices, kits, and systems are also provided.

### CROSS-REFERENCE TO RELATED APPLICATIONS

This application is the national stage entry of International Patent Application No. PCT/US2022/017083, filed on Feb. 18, 2022, and published as WO 2022/178313 A1 on Aug. 25, 2022, which claims the benefit of U.S. Provisional Patent Application Ser. No. 63/150,907, filed Feb. 18, 2021, both of which are hereby incorporated by reference herein in their entireties.

### BACKGROUND

In recent years, small extracellular vesicles (sEVs or exosomes) have emerged as important mediators of cell-to-cell communication with noted roles in regulating both physiological and pathological processes. These nanosized-vesicles (typically about 30-150 nm in diameter) are shed by every cell type and carry a wealth of cellular information including RNAs, lipids and proteins. Because of their abundance and stability in all biofluids, these vesicles hold promise as biomarkers for the diagnosis, prognosis and treatment efficacy for various diseases, conditions, and disorders, including infectious diseases and metabolic diseases associated with obesity, among many other examples.

To illustrate, small non-coding RNAs (ncRNAs) are the most abundant RNA component inside sEVs. MicroRNAs (miRs) are the most studied small ncRNAs, because of their ability to inhibit the translation of nearly 60% of all human genes. The identification of miRs inside sEVs also known as (ExomiRs) has revolutionized the field, as the presence of a miR inside sEVs yields information about miRs that are, for example, upregulated in the sEV parental cell. Upregulation of a miR in a particular cell type can be an indicator of cellular health. However, enthusiasm for the use of sEVs as a tool for liquid biopsies has been tempered by issues with the isolation and analysis methods and the uncertainty about the cellular source of the sEVs. The inter-individual differences in serum sEV cargo may reflect altered ratios of sEV sources rather than a true change in exosomal material from a single cell source.

To further illustrate in the context of infectious diseases, coronavirus disease 2019 (COVID-19) is caused by the Severe Acute Respiratory Syndrome Coronavirus-2 (SARS-CoV-2). As of September 2020, infected patients were present in almost all countries/regions of the world, with almost 30 million cases worldwide and nearly 950,000 deaths. While the outbreak began in China, the USA has now surpassed every other country in terms of infected individuals and total deaths, with continuing spread. Moreover, many scientists believe COVID-19 will become a seasonal condition, much like influenza. As such, COVID-19 will continue to be a global public health threat for the foreseeable future. Tens of thousands of COVID-19 diagnostic tests are performed worldwide each day. However, there are several limitations of the current testing platforms. While qPCR based COVID-19 testing from nasopharyngeal (N.P.) swabs is very effective, this method exposes healthcare workers to possible infections, which necessitates a constant supply of PPE. Furthermore, the turnaround time for test results in some parts of the USA continues to hinder efforts to contain viral spread. On the other hand, antibody tests can be performed quickly using a blood sample. However, specificity and sensitivity have been a major concern with antibody-based testing. In addition to these limitations, asymptomatic and pre-symptomatic cases are considered a major source of widespread COVID-19 infection. These individuals are likely not tested as they have no symptoms, and current tests may not accurately diagnose a patient early in the course of their infection. Moreover, pre-existing COVID-19 diagnostic tests typically cannot be done at home at low cost.

In view of the foregoing, it is apparent that there is an urgent need for additional methods of detecting diseases, including infectious diseases.

### SUMMARY

The present disclosure relates, in certain aspects, to methods, biosensor devices, kits, and systems of use in detecting various diseases, conditions, and disorders. In some embodiments, for example, the present disclosure provides methods of detecting various etiologic agents of infectious diseases, such as severe acute respiratory syndrome coronavirus-2 (SARS-CoV-2). Among other advantages, the technology disclosed herein provides, in certain embodiments, for the multiparametric detection of viral RNA (vRNA), including Spike (S) and Nucleocapsid (N) protein-coding regions, plus a panel of host EV microRNAs (exomiRs) that reveals infection even when viral loads are below detectable limits. This approach, for example, also decreases false-positives/negatives, the major limitation of antigen/antibody tests. Some embodiments provide a biosensor device that uses simple bind-elute microfluidics for sample separation followed by electrical, probe-based detection. In some embodiments, results are transferred to a healthcare provider's and/or patient's smart device within about 3 hours of less of taking the test. Moreover, in some aspects, the present disclosure provides methods and a related platform, Immunocapture of Cell-specific Exosomes (ICE) that typically involves first isolating cell-type specific small extracellular vesicles (sEVs or exosomes) from a given sample and evaluating the status of one or more miRNA(s) from those isolated cell-type specific small extracellular vesicles (e.g., to confirm that they contained the upregulated and/or downregulated miRNA(s) that are associated with damage in an exosome parent cell). These and other aspects will be apparent upon a complete review of the present disclosure, including the accompanying figures.

In one aspect, the present disclosure provides a method of detecting a disease, condition, or disorder in a subject. The method includes (a) obtaining a set of ribonucleic acid (RNA) molecules from a population of exosomes in a biological sample obtained from the subject, and (b) detecting a plurality of target RNA molecules corresponding to a selected set of cell-specific exosomal RNA molecules in the set of RNA molecules to generate a target RNA molecular profile for the sample. The method also includes (c) determining that the target RNA molecular profile substantially matches a reference RNA molecular profile that correlates with the disease, condition, or disorder in a subject and/or using at least one algorithm that predicts a likelihood that the target RNA molecular profile correlates with the disease, condition, or disorder in a subject, thereby detecting the the disease, condition, or disorder in the subject.

In one aspect, the present disclosure provides a method of detecting severe acute respiratory syndrome coronavirus-2 (SARS-CoV-2) in a biological sample. The method includes (a) obtaining a set of ribonucleic acid (RNA) molecules from a population of exosomes in the sample, (b) detecting a plurality of target RNA molecules corresponding to RNA molecules listed in Table 1 in the set of RNA molecules to generate a target RNA molecular profile for the sample, and (c) determining that the target RNA molecular profile substantially matches a reference RNA molecular profile that correlates with a SARS-CoV-2 infection in a subject and/or using at least one algorithm that predicts a likelihood that the target RNA molecular profile correlates with a SARS-CoV-2 infection in a subject, thereby detecting the SARS-CoV-2 in the biological sample. Optionally, confirmatory tests are performed to confirm positive SARS-CoV-2 infection test results.

In some embodiments, the method includes detecting all of the target RNA molecules corresponding to the RNA molecules listed in Table 1 in the set of RNA (i.e., microRNAs and exomiRs) molecules to generate the target RNA molecular profile for the sample. In some embodiments, the method includes determining a quantity (e.g., expression levels (upregulation/downregulation)) of one or more of the plurality of target RNA molecules corresponding to the RNA molecules listed in Table 1 in the sample. In some embodiments, the biological sample comprises a blood sample, a plasma sample, a serum sample, a nasopharyngeal sample, or a saliva sample.

In some embodiments, the method includes obtaining the biological sample from a test subject (e.g., via finger-pricked blood, swab (nasal (NP), saliva, etc.), or the like). In some embodiments, the method includes prognosing a likely outcome for the test subject based upon detecting the SARS-CoV-2 in the biological sample. In some embodiments, the method includes administering one or more therapies to the test subject based upon detecting the SARS-CoV-2 in the biological sample. In some embodiments, step (c) comprises using additional clinical data (e.g., symptomatic/asymptomatic, qPCR-determined viral load, biological sex, age (including pediatric populations), comorbidities, and/or the like) for the subject to detect the SARS-CoV-2 in the biological sample. In some embodiments, the target RNA molecular profile is indicative of a severity of the SARS-CoV-2 infection in the subject. In some embodiments, the algorithm comprises a machine learning algorithm.

In one aspect, the present disclosure provides a biosensor device that includes at least one separation module having a body structure comprising one or more fluidic channels disposed at least partially in the body structure, which separation module is configured to substantially separate ribonucleic acid (RNA) molecules from other components in a biological sample when the biological sample is introduced into the fluidic channels. The biosensor device also includes at least one biosensor module operably connected to the separation module, which biosensor module comprises at least one binding area that fluidly communicates with the fluidic channels, which binding area comprises a set of bioreceptors that are configured to bind a plurality of cell-specific exosomal RNA molecules when the cell-specific exosomal RNA molecules from the biological sample are in the binding area.

In one aspect, the present disclosure provides a biosensor device that includes at least one separation module having a body structure comprising one or more fluidic channels disposed at least partially in the body structure, which separation module is configured to substantially separate ribonucleic acid (RNA) molecules from other components in a biological sample when the biological sample is introduced into the fluidic channels. The biosensor device also includes at least one biosensor module operably connected to the separation module, which biosensor module comprises at least one binding area that fluidly communicates with the fluidic channels, which binding area comprises a set of bioreceptors that are configured to bind a plurality of target RNA molecules corresponding to RNA molecules listed in Table 1 when the ribonucleic acid (RNA) molecules from the biological sample are in the binding area.

In some embodiments, the set of bioreceptors is configured to bind all of the target RNA molecules corresponding to the RNA molecules listed in Table 1. In some embodiments, the biosensor device includes one or more nodes/anti-nodes positioned, or positionable, within sensory communication of the fluidic channels. In some embodiments, the biosensor device includes at least one acoustic-focusing transducer positioned, or positionable, within sensory communication of the fluidic channels. In some embodiments, the biosensor device includes at least one nanoporous membrane disposed at least partially in one or more of the fluidic channels. In some embodiments, the biosensor module comprises one or more field-effect transistors (FETs). In some embodiments, the FETs comprise operably connected carbon nanotubes (CNTs). In some embodiments, the biosensor module is complementary metal oxide semiconductor (CMOS) compatible. In some embodiments, the biosensor device comprises at least one bind-elute matrix. In some embodiments, the biosensor device disclosed herein is included as a component of a kit.

In one aspect, the present disclosure provides a system that includes the biosensor device as described herein. The system also includes a controller operably connected, or connectable, to the biosensor device, which controller comprises, or is capable of accessing, computer readable media comprising non-transitory computer executable instructions which, when executed by at least one electronic processor, perform at least: effecting separation of the RNA molecules from the other components in the biological sample when the biological sample is introduced into the fluidic channels; detecting binding of the target RNA molecules to the set of bioreceptors when the target RNA molecules are bound to the set of bioreceptors to generate a target RNA molecular profile for the sample; and determining that the target RNA molecular profile substantially matches a reference RNA molecular profile that correlates with a SARS-CoV-2 infection in a subject and/or using at least one algorithm that predicts a likelihood that the target RNA molecular profile correlates with a SARS-CoV-2 infection in a subject.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, and related systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 schematically shows exemplary methods steps according to one embodiment. As shown, the method includes a sample collection step in which, for example, 100 μl of blood or a nasal (NP) swab or saliva are taken. The method also includes a sample processing and characterization step in which the sample is processed into an exomiR sample using a fully automated platform. The exomiR sample is then characterized using the same automated platform. The method also includes data analysis or results viewing step in which the exomiR and viral gene data is sent to a server where a data analysis pipeline is applied to the data to produce diagnostically certain results, which are then to a user at a mobile device, such as a smart phone.

FIG. 2 schematically shows a flow chart that illustrates method steps according to some embodiments.

FIG. 3 schematically shows modules of an exemplary system according to one embodiment.

FIG. 4 schematically depicts elements of a data analysis (bioinformatics and machine learning) pipeline according to one embodiment.

FIGS. 5A-5D show the characterization of EVs obtained by bind-elute and regular SEC. Human blood plasma was processed with SmartSEC (bind-elute matrix) and Izon qEV SEC (70 nm cutoff). SmartSEC matrix was eluted three times (F1-3). 15 fractions were collected from regular SEC and pooled into fractions 7-9 (F1, EV-enriched), 10-12 (mixed protein and EV), and 13-15 (protein-enriched). A) Particle (nanoFCM), protein (microBCA), and ratios show roughly similar recovery with the two methods, although SEC provides a slight advantage in protein removal from EVs. B) EV marker TSG101 (other EV markers including tetraspanins were also assessed, not shown) is retained by SmartSEC, while HDL and L/VLDL markers are depleted in two trials. Albumin and cellular markers were also depleted (not shown). C) Electron micrograph of predominantly 30-100 nm particles eluted from SmartSEC matrix. D) Size profiles (nanoFCM) of three elutions from SmartSEC.

FIGS. 6 A and B show single-particle detection of SARS-CoV-2 mimics by SP-IRIS/Fluorescence. A) Control EVs have high expression of tetraspanins CD81, CD63, and CD9 by both label-free imaging of captured particles and fluorescence detection, but little background of SARS-CoV-2 detection. B) Engineered EVs with affinity-attached Spike protein are no longer efficiently captured by anti-tetraspanin antibodies, but are readily captured and detected by either anti-Spike reagents (e.g., D003, MM43) or biotin reagents. CD9 is also detected in these captured virus-like particles. We will use this system to generate non-infectious and well-characterized standards for our SARS-CoV-2 assays.

FIG. 7 shows a comparison between SARS-CoV-2 infected and uninfected control on small RNA-Seq. Volcano plot shows an insignificant finding on the differential expression of exomiRs between the SARS-CoV-2 infected and uninfected control. The observed relative difference (di) represented on the Y-axis is almost identical with the expected relative difference (di) represented on the X-axis.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administering: As used herein, the terms “administering” and “administration” refer to any method of providing a pharmaceutical preparation or other treatment to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

Bind: As used herein, “bind,” in the context of molecular detection, refers to a state in which a first chemical structure (e.g., a microRNA, a SARS-CoV-2 RNA, etc.) is sufficiently associated a second chemical structure such that the association between the first and second chemical structures can be detected.

Bioreceptor: As used herein, “bioreceptor” refers to a chemical structure that receives or binds biochemical structures (e.g a microRNA, a SARS-CoV-2 RNA, etc.).

Communicate: As used herein, “communicate” refers to the direct or indirect transfer or transmission, and/or capability of directly or indirectly transferring or transmitting, something at least from one area to another area.

Detecting: As used herein, “detecting,” “detect,” or “detection” refers to an act of determining the existence or presence of one or more target analytes (e.g., pathogenic particles, such as severe acute respiratory syndrome coronavirus-2 (SARS-CoV-2) particles) in a sample.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), support vector machines, decision trees (e.g., recursive partitioning processes such as CART —classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Pathogen: As used herein, “pathogen” or “pathogenic particle” refers to anything that can produce a disease, condition, or disorder in a subject. In some embodiments, a pathogen includes an infectious microorganism or agent, such as a bacterium, virus, viroid, protozoan, prion, or fungus.

Sample: As used herein, “sample” means anything capable of being analyzed using a device or system disclosed herein. Exemplary sample types include environmental samples and biological samples (e.g., biofluids or the like). In some embodiments, samples include a blood sample, a plasma sample, a serum sample, a nasopharyngeal swab viral transport media (VTM), or a saliva sample.

Severe Acute Respiratory Syndrome Coronavirus-2: As used herein, “severe acute respiratory syndrome coronavirus-2” or “SARS-CoV-2” refers to the coronavirus that emerged in 2019 to cause a human pandemic of an acute respiratory disease, now known as coronavirus disease 2019 (COVID-19).

Specifically Bind: As used herein, “specifically bind,” in the context of pathogen detection, refers to a state in which substantially only target chemical structures (e.g., a target microRNA, a target SARS-CoV-2 RNA, etc.) are sufficiently associated with a corresponding or cognate binding agent, to the exclusion of non-target chemical structures, such that the association between the target chemical structures and the binding agent can be detected.

System: As used herein, “system” in the context of analytical instrumentation refers a group of objects and/or devices that form a network for performing a desired objective.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a respiratory disease, disorder, or condition, is going to receive a therapy for a respiratory disease, disorder, or condition, and/or has received at least one therapy for a respiratory disease, disorder, or condition.

Therapy: As used herein, “therapy” or “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

### DETAILED DESCRIPTION

Various types of ribonucleic acids (RNAs) can be used to detect a wide array of diseases, conditions, and disorders. Circulating miRNAs, for example, are a new class of biomarkers and have been studied extensively for their diagnostic, predictive, or prognostic roles in malignancies and a wide range of non-neoplastic diseases. Accordingly, in some aspects, the present disclosure provides methods and platforms (denoted as Immunocapture of Cell-specific Exosomes (ICE) in some embodiments) that typically involve isolating cell-type specific small extracellular vesicles (sEVs or exosomes) and detecting one or more RNA molecules (e.g., viral RNA (vRNA), host EV microRNAs (exomiRs), and the like) obtained from the cell-type specific small extracellular vesicles as part of the diagnosis or other evaluation of a given disease, condition, and disorder.

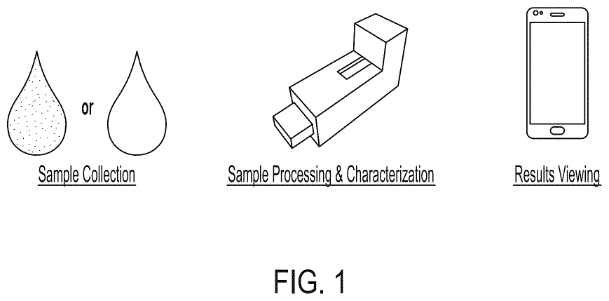

Infectious diseases, for example, pose significant public health risk. To illustrate, there is great urgency to curb the spread of SARS-CoV-2 and to treat infected patients in the U.S. and worldwide. The situation necessitates an affordable, rapid, accurate, sensitive and effective test in order to control this pandemic. Indeed, there are qPCR and antibody-based SARS-CoV-2 tests available, but current turnaround times for qPCR results do not allow for adequate control of disease spread. Antibody-based testing is faster, but the false positive/negative rates are too high. Accordingly, in certain aspects, the present disclosure provides a fully automated SARS-CoV-2 testing platform which is low-cost (˜$20 or less/test), accurate, sensitive, rapid (result within 3 hours of taking a test), and, practical. In some embodiments, the device is optimized to analyze blood and nasopharyngeal (NP) swab samples that an individual can collect at home, without risking transmission to healthcare workers. In some of these embodiments, the patient sample is processed using a fully automated platform and data are analyzed through an application that can be downloaded to any smart device (FIG. 1). Typically, the platform disclosed herein detects viral RNA, including the spike (S) glycoprotein and nucleocapsid (N) protein encoded regions as well as a panel of microRNAs (exomiRs) encapsulated within exosomal or small extracellular vesicles (sEVs). This approach significantly decreases false positive and false negative results, which is a major limitation of many antigen-based test, and provides insights into disease severity based on exomiR profiling. These and other aspects will be apparently upon complete review of this disclosure.

EVs have emerged as important mediators of cell-to-cell communication, regulating both physiological and pathological processes. Most abundant in the 30-150 nm diameter range, (“small EVs,” sEVs), they are released by cells from endosomal compartments (as “exosomes”) and from the cell surface (as “ectosomes” or “microvesicles”). Abundant and stable in biofluids, EVs carry a wealth of cellular information including RNAs, lipids and proteins that can be traced back to the cell of origin. Because SARS-CoV-2, is a type of “highjacked” EV and within the size range of sEVs, methodologies developed for sEV separation and characterization, as described herein, can be used for SARS-CoV-2 detection.

In general, effective viral diagnostics involve separation of particles from the high background of biofluid RNA signals and are not limited to infectious virions, but also takes into account host EV RNA such as exomiRs and vRNA packaged into non-virion EVs. The separation technology disclosed herein is rapid and cheap, using a bind-elute matrix in some embodiments. Typically, the readout is hybridization-based electrodetection that avoids downsides of PCR amplification. In some embodiments, the methods disclosed herein detect exomiRs such as miR-146a (markedly upregulated) and miR-16 (downregulated) that are differentially regulated in SARS-CoV-2 infection in blood (despite limited viral RNA detection) and NP swab VTM (with readily detected viral RNA). Per RNAseq and qPCR-confirmed data, the present disclosure includes a hybridization panel that detects 22 exomiRs along with three viral RNA regions. Bind-elute separation and electrical detection are combined in a small, home user-friendly device optimized for finger-prick blood, saliva, and NP swab fluid in some embodiments.

By way of additional background, (EVs) are released from most cell types. Originating at the plasma membrane (as “ectosomes,” “microvesicles”) or from the endosomal system (as “exosomes”), EVs have diameters that follow a power-law distribution. As such, they are most abundant in the 30-150 nm diameter range, where they are referred to as “small EVs” (sEVs) since biogenetic pathway is difficult to establish, particularly in complex biofluids. Minimal characterization of EVs involves detection of transmembrane or membrane-anchored proteins such as LAMPs and tetraspanins CD9, CD63, or CD81 (to demonstrate the presence of the lipid bilayer) and luminal cytoplasmic content such as TSG101 or syntenin. Technologies for separation and characterization of sEVs are well suited to SARS-CoV-2 diagnosis for two reasons. First, sEVs contain nucleic acids, enzymes, cytokines, and other bioactive compounds from the cell of origin, and EVs and their cargo can be modulated by the physiological state of the cell. EVs can thus serve as biomarkers of disease. Second, enveloped viruses such as SARS-CoV-2 share size, biogenesis, and other characteristics with host sEVs and can be considered a type of “highjacked” EV. Studies using both blood plasma and NP swab VTM have shown that SARS-CoV-2 infection regulates sEV cargo such as miRNAs and that methods for separation of EVs allow detection of viral RNA (vRNA) targets, especially from NP. It is also possible to detect asymptomatic and pre-symptomatic patients by examining sEV cargo. Furthermore, examining the sEV cargo can also help determine what types of acute reactions are occurring in the patient, such as an excessive inflammatory response, which could inform the care team about the best possible treatment options for the patient. Therefore, by combining measurements of host EV cargo and vRNAs, it is possible to determine the presence of SARS-CoV-2 infection (diagnosis) and to prognose disease course. In certain embodiments, the methods disclosed herein use sEV technologies to detect both the alterations of sEV host cargo due to SARS-CoV-2 infection as well as the presence of SARS-CoV-2 vRNAs.

SARS-CoV-2 attaches to the cell membrane using the S-protein region. Endocytosis and/or fusion mechanisms result in internalization of the virus into the host cell. The virus then uses host cell machinery to replicate itself in the cytoplasm of the host cell. Using cellular Golgi and Endoplasmic Reticulum intermediate complexes, the virus particles are packaged and eventually released as enveloped virions (similar to host sEVs) that subsequently infect more cells. Therefore, isolating EVs from an infected individual can enrich for viral particles and enhance detection. Based on several reports, COVID-19 is not considered a bloodborne infectious disease. For this reason, in blood plasma, we have found that signals for vRNAs are very low or even undetectable (again illustrating a need for examining host responses as well as viral molecules). In contrast, we can readily detect these RNAs within the sEV fraction from, for example, NP swab samples from the same individuals (Ct values ˜18). Studies have shown that protein and RNA fragments of the virus can be detected in viral particles in single sEVs. In some embodiments, using a fully automated and economical platform, the blood-sEV sample is used, as a blood sample is easy to obtain at home with, for example, a finger prick. If the sEV cargo miRNA (exomiR) profile correlates with the SARS-COV-2 infection profile, then the individual can immediately perform the second test using the sEV fraction from an NP swab (or saliva) that can also be collected at home in some embodiments. In these embodiments, this second test will confirm the presence of actual SARS-CoV-2 nucleic acid, such as the S-, N1- and N2-region vRNAs. The low cost of the tests disclosed herein make it easy for anyone to perform both tests, if necessary. Additionally, results are typically delivered rapidly (3 hours or less) without exposing healthcare workers or the public to the virus.

To further illustrate, the SARS-CoV-2 detection methods of the present disclosure include numerous advantageous aspects. For example, combining the exomiR panels with SARS-CoV-2 RNA targets, as described herein, improve detection of SARS-CoV-2, including asymptomatic and pre-symptomatic cases. Pediatric cases are also a major concern, as many studies have shown that infected children are less likely to be symptomatic than adults. The minimally invasive, low-cost tests of the present disclosure can be done frequently and rapidly in all population cohorts to allow immediate interventions to curb viral transmission. Sample collection is also straightforward. In some embodiments, for example, similar to testing glucose levels using finger sticks, the SARS-CoV-2 test can be readily performed at home, reducing the risk of transmission and the need for extensive PPE supplies for testing. Studies of exomiR cargo and vRNA presence in large cohorts of COVID-19 patient samples was used to generate profiles that, for example, indicate a mild or severe infection, helping doctors understand which patients may require hospitalization, and which treatments may be most effective for the patient based on how they are responding to the infection. This is especially valuable information for those populations with pre-existing conditions that are more likely to require hospitalization and suffer severe consequences with SARS-CoV-2 infection. Machine learning approaches are also used to further enhance the utility of this technology.

Moreover, some embodiments completely automate the sample processing steps from whole biofluid processing with either a bind-elute matrix or an acoustics-integrated, microfluidics-based to RNA isolation to total exomiR/vRNA detection is a diagnostic approach. Some embodiments involve the integration of carbon nanotubes (CNTs) and field-effect transistors (FETs) into a microRNA biosensor chip with high sensitivity, specificity, accuracy, and reproducibility. By coupling the target nanomaterial with electronics, in these embodiments, the platform is able to achieve exceptional measures of performance such as a limit of detection of 1 attomole. Furthermore, the use of CNTs and FETs allows the biosensing chip to be label-free, pushing the technology further away from conventional nucleic acid analysis technologies in these embodiments. Also, the biosensor chips disclosed herein typically do not involve amplification steps or expensive optics. Instead, miRNA/vRNA hybridization results are detected by an electrical reader in these embodiments. In some embodiments, the methods and related aspects disclosed herein for EV/viral separation and characterization include the latest cutting-edge single-particle technologies, such as nano-flow cytometry, SP-IRIS, microfluidic resistive pulse sensing, and single-molecule RNA detection.

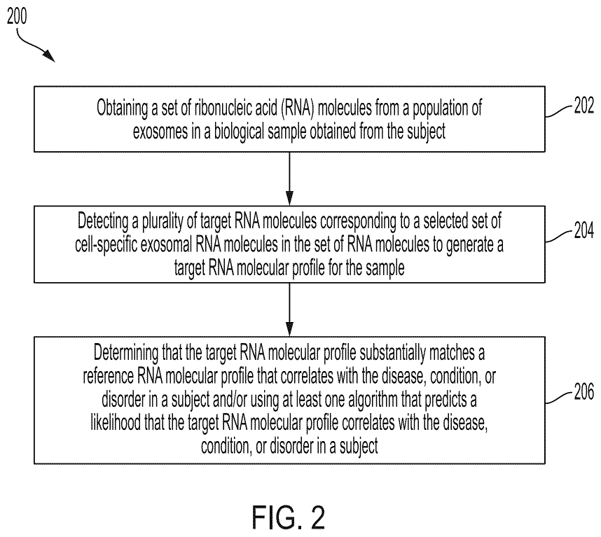

To illustrate, FIG. 2 schematically shows a flow chart that illustrates method steps of detecting a disease, condition, or disorder according to some embodiments. As shown, method 200 includes obtaining a set of ribonucleic acid (RNA) molecules from a population of exosomes in a biological sample obtained from the subject (step 202). Method 200 also includes detecting a plurality of target RNA molecules corresponding to a selected set of cell-specific exosomal RNA molecules in the set of RNA molecules to generate a target RNA molecular profile for the sample (step 204). In addition, method 200 also includes determining that the target RNA molecular profile substantially matches a reference RNA molecular profile that correlates with the disease, condition, or disorder in a subject and/or using at least one algorithm that predicts a likelihood that the target RNA molecular profile correlates with the disease, condition, or disorder in a subject (step 206).

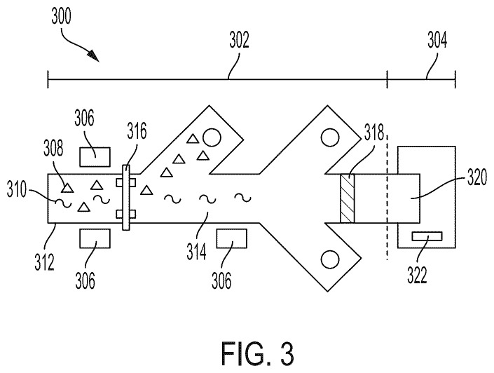

In some embodiments, the present disclosure provides biosensor devices and related systems for implementing the methods disclosed herein. In certain exemplary embodiments, the platform has a whole blood-specific configuration that incorporates multiple modules. Two modules of biosensor device 300 are shown in FIG. 3 and involve particulate/biomolecule separations (separation module 302) and target molecule detection (biosensor module 304). As shown, biosensor module 304 includes binding area 320 operably connected to field-effect transistor (FET) 322 for sensing, measuring RNA 310 separated from other exosomal debris 308 and result reporting (e.g., via Bluetooth-compatible interface). Separation module 302 includes body structure 312 comprising fluidic channels 314 disposed at least partially in body structure 312. Separation module 302 is configured to substantially separate RNA molecules 310 (e.g., exomiRs, vRNA, and/or the like) from other components 308 in a biological sample when the biological sample is introduced into fluidic channels 314. Biosensor module 304 is operably connected to the separation module 302. As shown, biosensor module 304 comprises binding area 320 that fluidly communicates with fluidic channels 314. Binding area 320 comprises a set of bioreceptors that are configured to bind a plurality of target RNA molecules 310 when the RNA molecules from the biological sample are in binding area 320. In some embodiments, target RNA molecules 310 correspond to one or more RNA molecules listed in Table 1.

As shown, the separation module 302 is for whole blood separation to obtain a cell-depleted plasma fraction (FIG. 3). Separation module 302 implements a particle separation method to obtain small particles in the sEV/exosome/virus size range. Separation module 302 also isolates RNA. In some embodiments, one or more modules operate with acoustofluidic systems (306) (e.g., acoustic-focusing transducers, etc.). Separation module 302 is also fitted with nanoporous membrane 318 to further separate the target particles or molecules (i.e., sEVs/viruses and RNA). Similarly, the system can easily be adapted for different separation technologies, such as the simple bind-elute chromatography option discussed above. The platform is fully mechanical and obviates the use of hazardous chemicals and kits for sample lysis and separation. Features such as channel length, acoustic input power, and flow rate can be adapted as needed for different biological sources or target entities. For example, the flow rate can be controlled by the geometry and design of the microfluidic channels. Manipulating the geometry, microfluidics, and acoustics surrounding each module can make the automated platform a viable tool for use in samples other than blood, such as the NP swab VTM and saliva samples or the like. Several aspects of these separation modules are discussed in more detail herein.

In some embodiments, the separation module 302 of the platform is configured to separate sEV-sized particulates from blood plasma samples. In these embodiments, acoustophoretic focusing is coupled with nanoporous membrane filtration. The fluidic channels of the biosensor devices can have essentially any configuration suitable for implementing the methods disclosed herein. A nanoporous membrane 318 excludes debris and particulates above a chosen size. The membrane is followed by another straight channel which marks the start of biosensor module 304.

In some embodiments, separation module 302 isolates RNA via a method called “acoustic lysing.” Acoustic transducers 306 that are perpendicular to the direction of flow produce an acoustic pressure that disrupts RNA-containing particles and frees RNAs from RNA-binding proteins. Following acoustic lysing, the free RNA-containing solution passes through a straight channel and passes through nanoporous membrane 318 to exclude larger particles. In some embodiments, biosensor device 300 includes one or more nodes/anti-nodes 316 positioned within sensory communication of fluidic channels 314.

The biosensor chip (e.g., biosensor module 304) employs electrical signaling systems. Some embodiments use field-effect transistors (FETs) (e.g., FET 322) because they can directly translate the interactions between target biological molecules and the FET surface into readable electrical signals. In the biosensor's FET, current typically flows along a semiconductor path, a channel that is connected to two electrodes: the source and the drain. The channel conductance between the source and the drain is modulated by a third, bottom-gated electrode that is given by the silicon wafer and is capacitively coupled through a thin dielectric layer. In this complementary metal oxide semiconductor (CMOS)-compatible FET biosensor, the channel is in direct contact with the environment. In conjunction with the ease of on-chip integration of device arrays and the cost-effective device fabrication, the surface ultrasensitivity makes the FET biosensors an extremely attractive technology.

To the further develop and complete the transduction element, the FET is integrated with a nanomaterial, carbon nanotubes (CNTs) in some embodiments. The biosensor chip's superior integration of transduction and recognition elements has resulted in a small, economical, fully automating RNA sensing platform with extremely high measures of performance and applicability in all possible point-of-care settings.

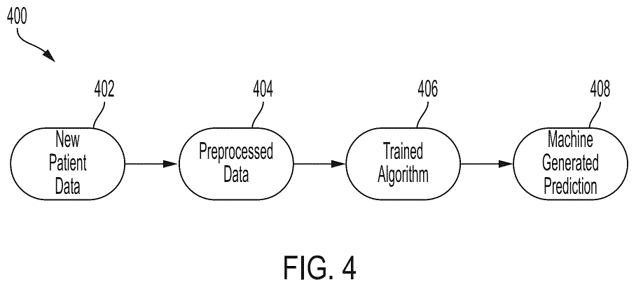

For diagnostics, a diagnostics database (new patient data 402) of data analysis pipeline 400 intakes data from healthy and diseased patients comprising of target exomiRs, their associated expression, stage, age, gender, race, ethnicity, place of birth, place of residence, preexisting conditions or diseases, genetic predispositions, and certain habits (i.e. smoking, drinking) (FIG. 4) in some embodiments. The purpose for the patient-related information is to establish demographical and population data behind the exomiR profile in order to increase diagnostic accuracy and sensitivity. With enough samples having been profiled and data being collected, the system is able to reach conclusions on a final diagnostic indication for a specific disease at a specific stage for a specific population. All of the noted information is stored within a diagnostic database where the data analysis is performed with goals of understanding patterns within the exomiR profile, demographical data, and disease. As also shown in FIG. 4, data analysis pipeline 400 also implements system components to generate preprocessed data 404, uses trained algorithm 406, and machine generated prediction functionality 408 to detect a given disease, condition, or disorder.

Upon receiving exomiR/vRNA expression data from a given biosensor device through the biosensor module 304 (FIG. 3), conversion software is typically applied to translate the received unit of measure for current, amperes, into a standard measure of concentration. Following the conversion, a normalization algorithm is typically applied to further remove data inconsistencies, reduce data redundancy, and improve data integrity. The normalized data is then typically put through a series of bioinformatic algorithms and processes, including statistical analysis, sequence alignment algorithms, correlation analysis algorithms, and clustering algorithms, among others. As a result of having an exhaustive list of diagnostic factors within a single profile, as described herein, numerous samples are generally tested to increase the pattern recognition and predictive capabilities of the platform. The platform is capable of intaking a given exomiR profile and labelling it to an exact disease/condition, stage, gender, race, ethnicity, demographic, and population or to a healthy patient with the same orders of information in some embodiments.

### EXAMPLES

### Example 1: Detection of COVID-19 Using a Panel of Exomirs and Vrnas

Preliminary Data

Characterization of EVs Isolated Using SmartSEC

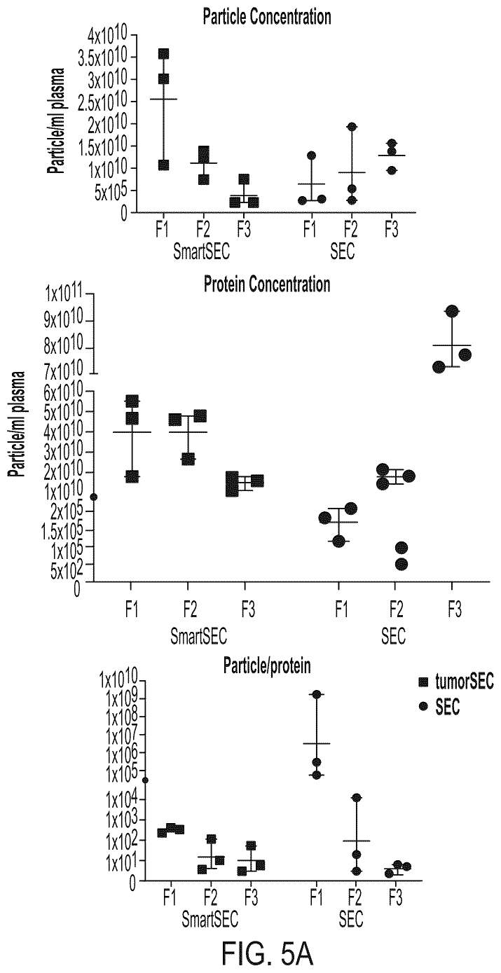

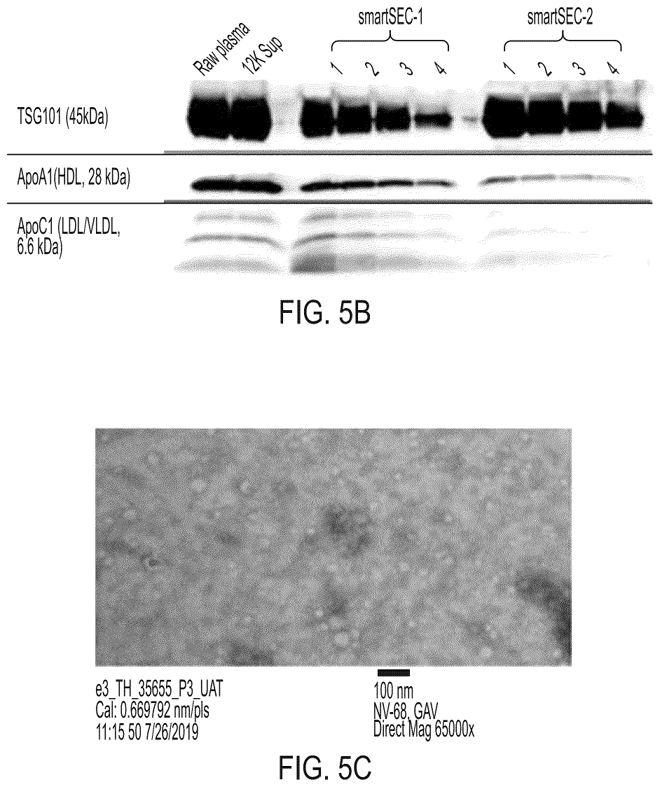

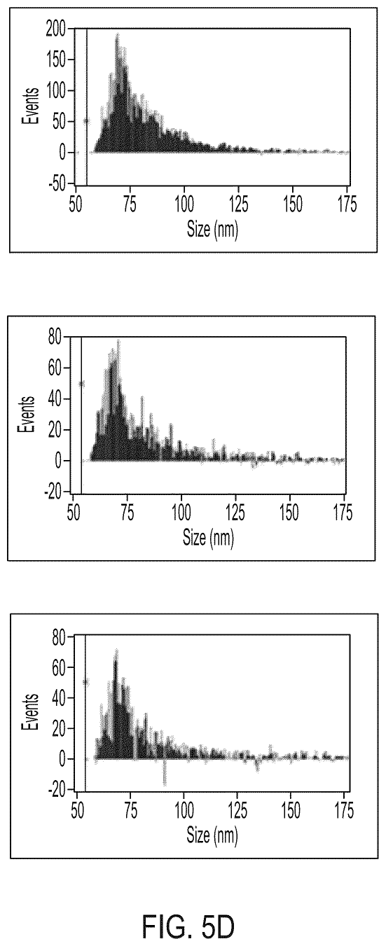

In this example, we used SmartSEC HT 96 well plates to separate the EV/virus fraction from plasma/NP swab VTM/saliva. The SmartSEC HT platform is based on the principle of size exclusion chromatography (SEC), but in contrast with traditional SEC, it operates as a “bind-elute” matrix, eliminating the need for fraction collection. It is thus scalable and high-throughput. The manufacturer is System Biosciences. The EVs separated with SmartSEC vs regular SEC were characterized, showing acceptable yield, purity, and size/marker profiles for this greatly simplified approach (FIG. 5).

Towards SARS-CoV-2 Reference Materials and vRNA Detection

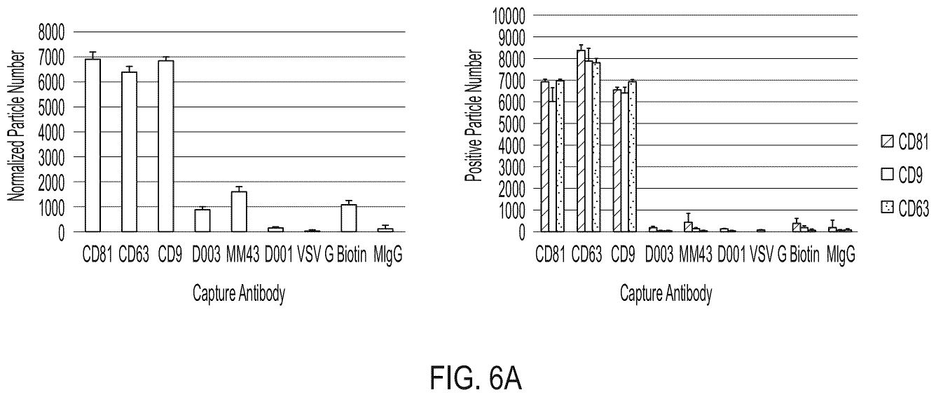

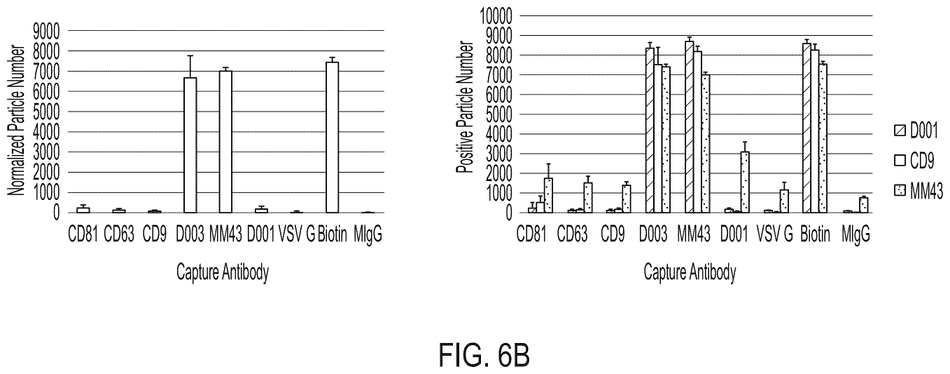

Preliminary studies showed no SARS-CoV-2 particles in the EV fraction from plasma samples, consistent with the lack of evidence for COVID-19 as a bloodborne disease. To generate standard materials that can be used to assess the specificity and sensitivity of our assays, SARS-CoV-2 mimics (virus-like particles, VLPs) were engineered in the scalable, suspension cell Expi-293F system. Recombinant SARS-CoV-2 soluble Spike was attached to EVs using biotin affinity. EVs/VLPs were analyzed at the single-particle level by NanoView “ExoView” SP-IRIS/fluorescence. As shown in FIG. 6, single SARS-CoV-2 mimics can be detected within an EV population.

For the experiments, viral RNA fragments and/or host miRNAs can be loaded into these VLPs to serve as reference materials. Indeed, we have also identified conserved S-, N1 and N2-protein encoding RNA of SARS-CoV-2 in EVs from NP swab VTM using regular qPCR.

Validation of the COVID-19 Panel of exomiRs and vRNAs

Bind-Elute sEV Separation from Plasma and NP Swabs

To obtain a high yield of highly pure sEVs in a high-throughput platform, we used the smartSEC 96 well plate (System Biosciences, CA). A total of 500 μl of either plasma or NP swab VTM was run through the bind-elute matrix.

Discovery Phase: ExomiR Profiles with COVID-19

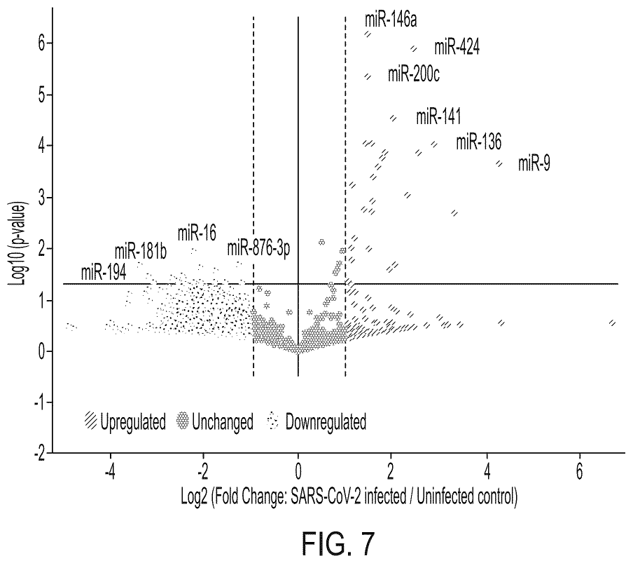

We used miRNA-Seq to identify mis-regulated exomiRs in plasma-derived sEVs of COVID patients (n=9) by comparing to the exomiR signatures of non-infected individuals (n=9) (FIG. 7). Altogether, 2001 known miRNAs were detected in the circulating exosomes from un-infected and COVID-19 patients by sequencing. We followed the QIAseq miRNA Library Kit (Qiagen) protocol, and sequencing was performed on the NextSeq 550 sequencing platform. Qubit (Thermo Scientific) and qPCR (using standard Illumina primers) were used to perform the quality control assessment of amplicons before sequencing, as described previously. From each sample, an average of 7.3 million reads were obtained, and approximately 16.4% of reads mapped to the human genome. From our comprehensive list of exomiRs, 32 were observed to be upregulated (>1.5 fold) and 46 exomiRs were downregulated in all of the COVID-19 patients.

Validation Phase: Dysregulated ExomiRs in COVID-19 Positive Plasma Samples

We performed qPCR to validate 78 miRNAs (32 upregulated and 46 downregulated) from the sEV fraction of plasma samples. In this phase we used a separate cohort of COVID-19 patients (n=30) and uninfected individuals (n=15). We validated 16 upregulated and 20 downregulated exomiRs from the miRNA-Seq list in COVID-19 μlasma sEVs.

Validation Phase: Dysregulated ExomiR in COVID-19 Positive NP Swab Samples

We performed qPCR to validate 78 miRNAs (32 upregulated and 46 downregulated) from the sEV fraction of NP swab samples. We used a separate cohort of COVID-19 patients from the initial study, but these were the same patients used for the plasma validation stage (n=30) and uninfected individuals (n=15). We validated 13 upregulated and 12 downregulated exomiRs from the miRNA-Seq list in COVID-19 patient NP swab sEV.

COVID-19 exomiR List

We have compared these two independent qPCR validation datasets, and identified a list of 22 exomiRs, which are strongly associated with SARS-CoV-2 infection (Table 1). In addition to detecting SARS-CoV-2 infection, exomiRs can also help us to assess specific COVID-19 complications, and aid in predicting which treatment options may most benefit the patient. Some or all of the identified exomiRs may also be altered in response to other viral infections or in the severe lung inflammation, such as with cystic fibrosis. To overcome this potential limitation, we assess the specificity of our responses in larger cohorts, but also confirm infection by testing for SARS-CoV-2-specific vRNA targets in the regions encoding the S and N proteins. Hence, we have developed a list of 25 targets—22 exomiRs and 3 SARS-CoV-2 vRNAs, for further validation.

TABLE 1
COVID-19 Panel

| | exomiR- | Regulation |
|---|---|---|
| 1 | 146a | Up |
| 2 | 424 | Up |
| 3 | 9 | Up |
| 4 | 136 | Up |
| 5 | 7 | Up |
| 6 | 187 | Up |
| 7 | 200c | Up |
| 8 | 1275 | Up |
| 9 | 29c | Up |
| 10 | 141 | Up |
| 11 | 15a/195 | Up |
| 12 | 145 | Down |
| 13 | 103a | Down |
| 14 | 885 | Down |
| 15 | 340 | Down |
| 16 | 194 | Down |
| 17 | 26a | Down |
| 18 | 181b | Down |
| 19 | 30b | Down |
| 20 | 142-3p | Down |
| 21 | 16 | Down |
| 22 | 876-3p | Down |
| 23 | S- | NA |
| 24 | N1- | NA |
| 25 | N2- | NA |

We developed a SARS-CoV-2 diagnostic device to detect these targets without the need for PCR amplification or expensive optics. Importantly, the detection platform can also accommodate more targets as needed based on the additional validation studies, in which we increase the number of samples for additional discovery (miRNA-Seq), validation from plasma and NP swab VTM, and expansion to saliva samples. This target list will be applicable (diagnostic and possible prognostic) for populations with different degrees of COVID-19 severity and co-morbidities.

### Example 2: Liquid Biopsy of Pancreatic Islet Cells and Endothelial Cells for Obesity-Induced Risk Assessment

### INTRODUCTION

We have utilized basic principles of sEV biogenesis to develop Immunocapture of Cell-specific Exosomes (ICE). Apart from the expression of specific exosomal markers, such as Lamp1, CD9, CD63, CD81 and TSG101, sEVs often express certain cell surface markers from their cell of origin. The tetraspanins, CD9, CD63, CD81 and TSG101, are sEV surface markers that are expressed during the process of nanovesicle formation inside endosomes. Accumulation of excess cytosolic materials, such as miRs and proteins, results in a signal to the cell membrane, which stimulates the invagination of the cell membrane to form early endosomes. During that phase, cellular surface markers are also incorporated into the surface of early endosomes, which are transferred to the exosomal surface. Herein, we have identified three specific sEV surface markers that can be used to capture pancreatic islet, endothelial cell, and adipocyte specific sEVs. By combining these ICE platforms, we have developed a workflow where with a minimal volume of blood (˜1 ml), we can ascertain whether patients have obesity-induced damage to pancreatic islet and endothelial cells.

Results

ExomiR Profiles with Obesity

We used miRNA-Seq to identify upregulated exomiRs in plasma-derived sEVs of obese individuals (BMI>30, n=9) by comparing to the sEV miR signatures of healthy individuals (BMI<25, n=9). Altogether, 2041 different known miRNAs were detected in the circulating exosomes from healthy and obese individuals by sequencing. We observed miR-16-5p to be the most abundant exomiR in both study groups. From our comprehensive list of exomiRs, 32 were observed to be upregulated (more than 1.5 fold) in all of the obese individuals. Since a substantial proportion of the circulating exomiRs are derived from blood leukocytes and platelets, we next explored the cellular origin of these upregulated 32 exomiRs. Obesity is associated with pancreatic islet cell and endothelial cell dysfunction. Therefore, we attempted to establish the miRNA signatures of sEVs derived from pancreatic beta cells, endothelial cells and adipocytes.

Pancreatic Islet-Specific ICE

We cultured EndoC-BH1 human β-cells (Univercell Biosolutions) to identify and validate potential pancreatic islet cell derived sEV surface makers. We tested whether several known islet-specific proteins are expressed by sEVs isolated from the EndoC-BH1 culture media. Using flow cytometry, we validated that Glucagon-like peptide-1 receptor (GLP-1R) is expressed on the surface of the EndoC-BH1 sEVs (FIG. 2B). This allowed us to design a method for capturing pancreatic islet-specific sEVs from any given body fluid. We subsequently sequenced the miRs isolated from EndoC-BH1 sEVs.

Endothelial Cell-Specific ICE

Utilizing similar methods, we cultured primary HUVEC cells to identify and validate endothelial cell derived sEV surface makers. We initially tested several known endothelial cell specific proteins. Using flow cytometry, we validated that CD146 is expressed on the surface of sEVs secreted from HUVECs. We utilized CD146 to design a method for capturing endothelial cell sEVs from bodily fluids.

Adipocytes-Specific ICE

FABP4 (Fatty Acid Binding Protein 4).

Liquid Biopsy Platform for Obesity Risk-Assessment

In order to understand the origins of circulating exomiRs in the context of obesity, we developed an easy but innovative method to isolate cell specific exomiRs from plasma. As part of the overall workflow, we separated (i) pancreatic islet, (ii) endothelial cell, and (iii) adipocyte specific sEVs in a sequential manner from all sEVs that were isolated from 500 μl of human plasma. We established this order for the isolations based on the relative abundance of each circulating sEV population. For this workflow, we utilized plate bound antibodies against the surface proteins GLP-1R, CD146 and FABP4 to capture pancreatic islet, endothelial cell and adipocyte specific sEVs, respectively, from the plasma of obese (n=25) and healthy (n=25) subjects. The FluoroCet Exosome Quantitation Kit (SBI) was used to quantify the attached cell-specific sEVs.

A. Pancreatic Islet Damage Assessment

Because the majority of exosomes isolated from plasma originate from blood cells and platelets, we checked the expression of miR-451a and miR-223 in GLP-1R captured exosomes. We demonstrate that there is low expression of miR-451a and miR-223 compared to the expression of pancreatic islet-specific miR-375, which suggests that the ICE method is greatly enriching for islet-derived exosomes. In obese individuals, there is significantly higher expression of miR-29a-3p, miR-203a-3p, miR-194-5p, miR-29c-3p, miR-30a-5p, miR-148a-3p and miR-126-5p compared to healthy individuals when profiling pancreatic islet-derived sEVs. However, we did not observe any significant differences in the expression of miR-432, miR-199b-3p, miR-375, miR-148b-3p and miR-151a between obese and healthy individuals in the pancreatic islet-derived sEVs.

B. Endothelial Cell Damage Assessment

The sEV-fraction that did not attach to Plate 1 (pancreatic islet cell ICE plate) was subsequently incubated with Plate 2 (the endothelial cell-specific ICE plate). We extracted the miRNA fraction from sEVs that bound to Plate 2, and queried exomiR expression. We observed significantly higher expression of miR-92b-5p, miR-149-3p, miR-8078, and miR-193b-5p in the endothelial cell specific exosomes derived from the circulating exosomes of obese individuals. These endothelial specific exomiRs can be utilized as risk assessment markers and for monitoring the status of endothelial cell dysfunction due to obesity.

C. Adipocyte-Specific sEV Capture and Validation of Our Serial ICE Platform

Finally, to validate our serial ICE workflow, we incubated the unbound sEV fraction from Plate 2 with the adipocyte-specific ICE Plate 3. As expected, there are significantly more circulating adipocyte-specific sEVs in obese individuals compared to healthy individuals. We performed RNA-seq on the RNA extracted from bound sEVs from Plate 3. We detected 20 miRs which are known to be highly expressed in human fat cells (from the literature). We were also able to validate that all 20 of these miRs are markedly higher in obese individuals compared to healthy subjects. We performed this step to clearly validate our workflow.

### DISCUSSION

Circulating miRs have the potential to be robust early diagnostic and prognostic biomarkers for a disease. Cells that are stressed or damaged have altered miR profiles, and numerous studies have validated that certain miRs have pathogenic roles in these cells. Overexpressed miRs can be packaged into sEVs, which circulate in the blood. Therefore, extracting cell specific exosomes from the circulation and assessing their miR profiles can offer important information on the overall health of the sEV parental cell. Despite the potential to be a powerful tool for the early diagnosis of cellular damage, most of the miR early diagnostic manuscripts have yielded little clinical utility. This is in part due to the fact that few studies have considered the origin of circulating miRs, and whether elevated circulating miRs are actually derived from cells involved in the pathophysiology of a particular disease. It is known that one miR can have detrimental functions in one cell type, but completely different functions in another cell type. Therefore, when assessing circulating miRs as potential disease biomarkers, it is critical to identify their cellular source.

Exosomal miR (exomiR) are protected from RNases in the blood, making them attractive targets for disease biomarkers. This led us to develop a tool to capture cell-specific manner from the circulation. As circulating exomiRs are derived from various cell types, understanding their cellular lineage is a necessary step in order to define their involvement in disease etiology and progression. Our ICE technology facilitates the capturing of cell-specific sEVs from the circulation. In this study, we have highlighted a platform that utilizes a small volume of plasma (500 μl) that is subjected to sequential ICE protocols, which yields clinically relevant information about the overall health of multiple cell types in the context of obesity.

For proof-of-concept to establish the utility of this platform, we used an obesity group (BMI>30) and compared their blood samples with healthy subjects (BMI<25). The obese subjects in this study did not present with any symptoms of other clinical abnormalities at the time of blood collection. However, our platform detected that obesity itself is causing both pancreatic islet, as well as endothelial cell damage. It is well know that obesity is a major risk factor for type 2 diabetes and cardiovascular disease. Pancreatic islet cell damage can lead to the clinical symptoms of type 2 diabetes. Similarly, endothelial cell damage can lead to cardiovascular disease such as ischemic heart disease which is overtaking cancer as the leading cause of death in the Western World. We utilized an advanced but simple methodology to identify cell specific exomiRs, and present for the first time a list of promising exomiRs derived from three critical cell types (pancreatic islets, endothelial cells and adipocytes) in obesity. We used adipocyte-specific exomiRs as a validation step for our platform. A liquid biopsy approach to query the exomiRs derived from pancreatic islet and endothelial cells has the potential to assess cellular health in the context of obesity before clinical symptoms of diabetes or cardiovascular diseases develop.

Apart from the reason that obesity affects multiple cell types in human body, we used this model to establish a simple blood test for a health condition that has no overt clinical symptoms. Thereby giving a window of opportunity to modify the natural history of obesity related disease before irreversible end organ damage has occurred. Our platform can be used to monitor obese patients for endothelial and pancreatic cell dysfunction and provide an alternative way to stratify their risk of disease that precedes the development of disease symptoms, offering the opportunity for low cost (e.g behavioural changes) interventions and to focus on disease prevention.

Among the 32 upregulated exomiRs from the circulating sEV fraction, 7 exomiRs (hsa-miR-126-5p, hsa-miR-148a-3p, hsa-miR-29c-3p, hsa-miR-29a-3p, hsa-miR-151a-3p, hsa-miR-199b-3p, hsa-miR-148b-3p, miR-30a-5p) are highly expressed in both pancreatic beta cells and adipocytes; while miR-192-5p is expressed in both endothelial cells and adipocytes. A few exomiRs were exclusively expressed in pancreatic beta cells (miR-432, miR-203a-3p, miR-375, miR-194-5p), adipocytes (hsa-miR-27b-3p) and endothelial cells (miR-92b-5p, miR-149-3p, miR-8078, miR-193b-5p). These observations indicate that circulating exomiRs are derived from a variety of cell types and this can be utilized to monitor pathological conditions.

Methods

Plasma Collection

Human plasma samples studied were from a biobank resource of deceased multi-organ donors with appropriate informed consent from the family. Plasma samples (in EDTA tubes) were taken in theatre at time of organ donation. The cohort was divided into two groups of BMI >30 (n=25, mean age 45.5±10.4 years, mean BMI 34.5±5.1) and BMI <25 (n=25, mean age 42.9±14.2 years, mean BMI 23.5±2.9). All samples were from male donors and none were on oral hypoglycemics. Half the cohort were donors after brain stem death and the remaining were donors after cardiac death. This research has been conducted under the remit of the UK QUOD Consortium supported by NHS Blood and Transplant (NRES Committee North West —Greater Manchester Central, REC 13/NW/0017).

sEV-Enriched Fraction Isolation

The sEV fraction was isolated by differential centrifugation: first, 1000×g for 5 min at 4° C., after which the supernatant was spun at 2500×g for 15 min at 4° C. Next, the supernatant was collected and spun at 10,000×g for 30 min at 4° C. The supernatant was filtered using a 0.22 μm filter and then ultracentrifugation (100,000×g for 90 min) was performed. Approximately 200 μl of the lower fraction in the tubes was collected (as there is no visible pellet), and washed with sterile PBS, followed by a second round of ultracentrifugation (100,000×g for 90 min). Again, approximately 200 μl of the lower fraction in the tubes was used as the sEV-enriched fraction for the study.

Total Exosomal RNA Isolation

The MiRNeasy Serum/Plasma Advanced kit (Qiagen) was used to isolate RNA from the plasma sEVs fraction. Fragment Analyzer Systems (Agilent) was used to check the quality of and determine the concentration of small RNAs.

RNA-Sequencing from Total Exosomal RNA

We followed the QIAseq miRNA Library Kit (Qiagen) protocol, and sequencing was performed on the NextSeq 550 sequencing platform. Qubit (Thermo Scientific) and qPCR (using standard Illumina primers) were used to perform the quality control assessment of amplicons before sequencing, as described previously. From each sample, an average of 7.3 million reads were obtained and approximately 16.4% of reads mapped to the human genome.

ICE Assays for Pancreatic Islet, Endothelial Cell, and Adiopocyte-Specific sEV Capture

Plates with incubated with GLP-1R (pancreatic islet), CD-146 (endothelial cell), and FABP4 (adipocyte) antibodies overnight at 4° C. The plates were then washed three times. As shown in FIG. 4A, the sEV-enriched fraction was first incubated in the GLP-1R antibody coated plate (plate 1) overnight at 4° C. with gentle shaking (400 rpm). The liquid volume from plate 1 was then transferred to the CD-146 antibody coated plate (Plate 2), which was then incubated overnight at 4° C. with gentle shaking (400 rpm). Finally, the liquid volume from plate 2 was then transferred to the FABP4 antibody coated plate (Plate 3). This incubated overnight at 4° C. with gentle shaking (400 rpm).

qPCR Validation of Pancreatic Islet and Endothelial Cell Specific Risk Assessment miRNAs

Plates 1 and 2 were washed three times with wash buffer (Sterile PBS with 0.01% tween). The miRNA fraction was isolated and pre-amplified using the miScript Single Cell qPCR kit (Qiagen). The miScript SYBR Green PCR kit (Qiagen) was used to perform qPCR. miR specific primers were purchased from Qiagen. The PCR reactions were run using Quant Studio 5 (Thermo).

Validation of Attached sEVs During ICE

After lysing the exosomes attached to ICE plates, the activity of acetecylcholinesterase (AChE) was measured using the FluoroCet Exosome Quantitation Kit (SBI) per the company's instruction.

miRNA-Sequencing from the ICE Fraction

Adipocyte-specific sEVs were lysed in plate 3 wells, and samples were then sent to Firalis for further processing. All data was analyzed at Firalis.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting a disease, condition, or disorder in a subject, the method comprising:
(a) obtaining a set of ribonucleic acid (RNA) molecules from a population of exosomes in a biological sample obtained from the subject;
(b) detecting a plurality of target RNA molecules corresponding to a selected set of cell-specific exosomal RNA molecules in the set of RNA molecules to generate a target RNA molecular profile for the sample; and,
(c) determining that the target RNA molecular profile substantially matches a reference RNA molecular profile that correlates with the disease, condition, or disorder in a subject and/or using at least one algorithm that predicts a likelihood that the target RNA molecular profile correlates with the disease, condition, or disorder in a subject, thereby detecting the disease, condition, or disorder in the subject.

2. A method of detecting severe acute respiratory syndrome coronavirus-2 (SARS-CoV-2) in a biological sample, the method comprising:
(a) obtaining a set of ribonucleic acid (RNA) molecules from a population of exosomes in the sample;
(b) detecting a plurality of target RNA molecules corresponding to RNA molecules listed in Table 1 in the set of RNA molecules to generate a target RNA molecular profile for the sample; and,
(c) determining that the target RNA molecular profile substantially matches a reference RNA molecular profile that correlates with a SARS-CoV-2 infection in a subject and/or using at least one algorithm that predicts a likelihood that the target RNA molecular profile correlates with a SARS-CoV-2 infection in a subject, thereby detecting the SARS-CoV-2 in the biological sample.

3. The method of claim 2, comprising detecting all of the target RNA molecules corresponding to the RNA molecules listed in Table 1 in the set of RNA molecules to generate the target RNA molecular profile for the sample.

4. The method of claim 2, comprising determining a quantity of one or more of the plurality of target RNA molecules corresponding to the RNA molecules listed in Table 1 in the sample.

5. The method of claim 2, wherein the biological sample comprises a blood sample, a plasma sample, a serum sample, a nasopharyngeal sample, or a saliva sample.

6. The method of claim 2, comprising obtaining the biological sample from a test subject.

7. The method of claim 2, comprising prognosing a likely outcome for the test subject based upon detecting the SARS-CoV-2 in the biological sample.

8. The method of claim 2, comprising administering one or more therapies to the test subject based upon detecting the SARS-CoV-2 in the biological sample.

9. The method of claim 2, wherein step (c) comprises using additional clinical data for the subject to detect the SARS-CoV-2 in the biological sample.

10. The method of claim 2, wherein the target RNA molecular profile is indicative of a severity of the SARS-CoV-2 infection in the subject.

11. The method of claim 2, wherein the algorithm comprises a machine learning algorithm.

12. A biosensor device, comprising:
at least one separation module having a body structure comprising one or more fluidic channels disposed at least partially in the body structure, which separation module is configured to substantially separate ribonucleic acid (RNA) molecules from other components in a biological sample when the biological sample is introduced into the fluidic channels; and,
at least one biosensor module operably connected to the separation module, which biosensor module comprises at least one binding area that fluidly communicates with the fluidic channels, which binding area comprises a set of bioreceptors that are configured to bind a plurality of cell-specific exosomal RNA molecules when the cell-specific exosomal RNA molecules from the biological sample are in the binding area.

13. A biosensor device, comprising:
at least one separation module having a body structure comprising one or more fluidic channels disposed at least partially in the body structure, which separation module is configured to substantially separate ribonucleic acid (RNA) molecules from other components in a biological sample when the biological sample is introduced into the fluidic channels; and,
at least one biosensor module operably connected to the separation module, which biosensor module comprises at least one binding area that fluidly communicates with the fluidic channels, which binding area comprises a set of bioreceptors that are configured to bind a plurality of target RNA molecules corresponding to RNA molecules listed in Table 1 when the ribonucleic acid (RNA) molecules from the biological sample are in the binding area.

14. The biosensor device of claim 13, wherein the set of bioreceptors is configured to bind all of the target RNA molecules corresponding to the RNA molecules listed in Table 1.

15. The biosensor device of claim 13, comprising one or more nodes/anti-nodes positioned, or positionable, within sensory communication of the fluidic channels.

16. The biosensor device of claim 13, comprising at least one acoustic-focusing transducer positioned, or positionable, within sensory communication of the fluidic channels.

17. The biosensor device of claim 13, comprising at least one nanoporous membrane disposed at least partially in one or more of the fluidic channels.

18. The biosensor device of claim 13, wherein the biosensor module comprises one or more field-effect transistors (FETs).

19. The biosensor device of claim 18, wherein the FETs comprise operably connected carbon nanotubes (CNTs).

20. The biosensor device of claim 13, wherein the biosensor module is complementary metal oxide semiconductor (CMOS) compatible.

21. The biosensor device of claim 13, wherein the biosensor device comprises at least one bind-elute matrix.

22. A kit comprising the biosensor device of claim 13.

23. A system, comprising:
the biosensor device of claim 13; and,
a controller operably connected, or connectable, to the biosensor device, which controller comprises, or is capable of accessing, computer readable media comprising non-transitory computer executable instructions which, when executed by at least one electronic processor, perform at least:
effecting separation of the RNA molecules from the other components in the biological sample when the biological sample is introduced into the fluidic channels;
detecting binding of the target RNA molecules to the set of bioreceptors when the target RNA molecules are bound to the set of bioreceptors to generate a target RNA molecular profile for the sample; and,
determining that the target RNA molecular profile substantially matches a reference RNA molecular profile that correlates with a SARS-CoV-2 infection in a subject and/or using at least one algorithm that predicts a likelihood that the target RNA molecular profile correlates with a SARS-CoV-2 infection in a subject.
